(19) United States
(12) Patent Application Publication
(10) Pub. No.: US 2018/0298279 A1
(43) Pub. Date: Oct. 18, 2018

(54) SPIRALLY CONFIGURED CIS-STILBENE/FLUORENE HYBRID MATERIAL AND ORGANIC ELECTROLUMINESCENT DEVICE USING THE SAME

(71) Applicant: JUST ABOUT SHOWING CO., LTD., New Taipei City (TW)

(72) Inventor: Jen-Cherng LEE, New Taipei City (TW)

(21) Appl. No.: 15/630,425

(22) Filed: Jun. 22, 2017

(30) Foreign Application Priority Data
Apr. 13, 2017 (TW) 106112303

(51) Int. Cl. C09K 11/06; C07C 13/42

(52) U.S. Cl. CPC C09K 11/06; C07C 2603/32; C09K 2211/1011; C07C 13/42

(57) ABSTRACT

A spirally configured cis-stilbene/fluorene hybrid material is shown in formula (1),

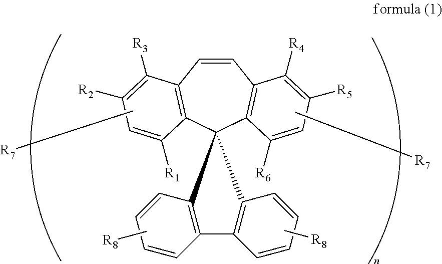

wherein n is 1, 2 or 3, R₁ to R₆ are independently selected from the group consisting of hydrogen atom, halogen atom, cyano group, alkyl group, cycloalkyl group, alkoxy group, amino group, haloalkyl group, thioalkyl group, silyl group, alkenyl group and aromatic ring group, R₈ is a hydrogen atom, tert-butyl group or aryl group, when n is 1, R₇ is a hydrogen atom, diarylamino group or 2-diarylamino-spirobifluorene group, when n is 2 or 3, R₇ is a hydrogen atom, diarylamino group or 2-diarylamino-spirobifluorene group.

### CROSS REFERENCE TO RELATED APPLICATIONS

This Non-provisional application claims priority under 35 U.S.C. § 119(a) on Patent Application No(s). 106112303 filed in Taiwan, Republic of China on Apr. 13, 2017, the entire contents of which are hereby incorporated by reference.

### BACKGROUND OF THE INVENTION

### Field of Invention

The present invention relates to an electroluminescent material and an electroluminescent device by using the same and, in particular, to a spirally configured cis-stilbene/fluorene hybrid material and an organic electroluminescent device by using the same.

### Related Art

With the advances in electronic technology, a light weight and high efficiency flat display device has been developed. An organic electroluminescent device becomes the mainstream of the next generation flat panel display device due to its advantages of self-luminosity, no restriction on viewing angle, low power conservation, simple manufacturing process, low cost, high response speed, full color and so on.

In general, the organic electroluminescent device includes an anode, an organic luminescent layer and a cathode. When applying a direct current to the organic electroluminescent device, holes and electrons are injected into the organic luminescent layer from the anode and the cathode, respectively. Charge carriers move and then transport into the organic luminescent layers because of the potential difference caused by an applied electric field. The resulting excited luminescent molecules (i.e., excitons) are generated and followed by the recombination of the electrons and the electron holes may lead to emission in the organic luminescent layer due to release the energy in the form of light.

Nowadays, the organic electroluminescent device usually adopts a host-guest emitter system. The organic luminescent layer disposed therein includes a host material and a guest material. The holes and the electrons are transmitted to the host material, and further transferred to the guest material to form excitons and then generate light. The guest material can be categorized into fluorescent material and phosphorescent material. Theoretically, the internal quantum efficiency can approach 25% by using appropriate fluorescent materials. Comparing with the phosphorescent materials, the fluorescent materials have longer shelf and device lifespan and lower cost.

Besides, the morphological defect of small molecule material, such as easy crystallization or easy cracking, restricts its application in flexible device. Moreover, the difficulty in purification of polymer materials leads to serious disadvantages in considerable decay of its device efficiencies and lifespan.

Accordingly, the present invention is provided a spirally configured cis-stilbene/fluorene hybrid material and an organic electroluminescent device by using the same which has advantages of excellent electronic, optical, optoelectronic perperties, thermal stability, film ductility and ease of purification.

### SUMMARY OF THE INVENTION

In view of the foregoing objectives, the invention provides a spirally configured cis-stilbene/fluorene hybrid material and an organic electroluminescent device by using the same. The spirally configured cis-stilbene/fluorene hybrid material has advantages of excellent film ductility, electronic, optical and optoelectronic properties, thermal stability, and ease of purification.

A spirally configured cis-stilbene/fluorene hybrid material, comprising a structure of the following General Formula (1).

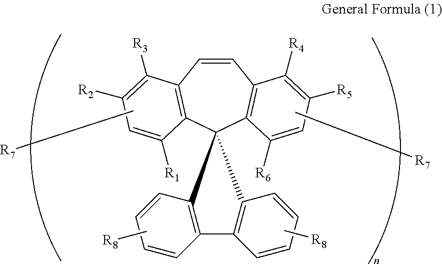

In the General formula (1), n is 1, 2 or 3, R₁ to R₆ are each independently selected from the group consisting of a hydrogen atom, halogen atom, cyano group, alkyl group, cycloalkyl group, alkoxy group, amino group, haloalkyl group, thioalkyl group, silyl group, alkenyl group and aromatic ring group, R₈ is a hydrogen atom, tert-butyl group or aromatic ring group. When n is 1, R₇ is 9,9′-spirobifluorene group, diarylamino group or 2-diarylamino-spirobifluorene group. When n is 2 or 3, R₇ is a hydrogen atom, diarylamino group or 2-diarylamino-spirobifluorene group.

Also, according to another embodiment of the present invention, an organic electroluminescent device is disclosed. The organic electroluminescent device comprises a first electrode layer, a second electrode layer and an organic luminescent unit. The organic luminescent unit is deposited between the first electrode layer and the second electrode layer. The organic luminescent unit has at least a spirally configured cis-stilbene/fluorene hybrid material as shown in General Formula (1).

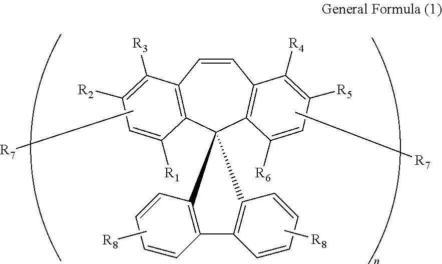

In the General formula (1), n is 1, 2 or 3, R₁ to R₆ are each independently selected from the group consisting of a hydrogen atom, halogen atom, cyano group, alkyl group, cycloalkyl group, alkoxy group, amino group, haloalkyl group, thioalkyl group, silyl group, alkenyl group and aromatic ring group, R₈ is a hydrogen atom, tert-butyl group or aromatic ring group. When n is 1, R₇ is 9,9′-spirobifluorene group, diarylamino group or 2-diarylamino-spirobifluorene group. When n is 2 or 3, R₇ is a hydrogen atom, diarylamino group or 2-diarylamino-spirobifluorene group.

In one embodiment, the alkyl group is selected from the group consisting of a substituted or unsubstituted straight-chain alkyl group with the carbon number of 1 to 6, and a substituted or unsubstituted branched-chain alkyl group with the carbon number of 3 to 6, the cycloalkyl group is a substituted or unsubstituted cycloalkyl group with the carbon number of 3 to 6, the alkoxy group is selected from the group consisting of a substituted or unsubstituted straight-chain alkoxy group with the carbon number of 1 to 6, and a substituted or unsubstituted branched-chain alkoxy group with the carbon number of 3 to 6, the amino group is selected from the group consisting of secondary amino group and tertiary amino group, the secondary amino group is an amino group having one aromatic ring substituent or having one C1-C6 straight-chain alkyl, branch-chain alkyl, or non-aromatic ring, the tertiary amino group is an amino group having two independent aromatic ring substituents or having two independent C1-C6 straight-chain alkyl, branch-chain alkyl, or non-aromatic ring, the haloalkyl group is selected from the group consisting of a substituted or unsubstituted straight-chain haloalkyl group with the carbon number of 1 to 6, and a substituted or unsubstituted branched-chain haloalkyl group with the carbon number of 3 to 6, the thioalkyl group is selected from the group consisting of a substituted or unsubstituted straight-chain thioalkyl group with the carbon number of 1 to 6, and a substituted or unsubstituted branched-chain thioalkyl group with the carbon number of 3 to 6, the silyl group is selected from the group consisting of a substituted or unsubstituted straight-chain silyl group with the carbon number of 1 to 6, and a substituted or unsubstituted branched-chain silyl group with the carbon number of 3 to 6, the alkenyl group is selected from the group consisting of a substituted or unsubstituted straight-chain alkenyl group with the carbon number of 2 to 6, and a substituted or unsubstituted branched-chain alkenyl group with the carbon number of 3 to 6.

In one embodiment, the spirally configured cis-stilbene/fluorene hybrid material can be represented by following chemical formula (1), chemical formula (2), chemical formula (3), chemical formula (4), chemical formula (5) or chemical formula (6).

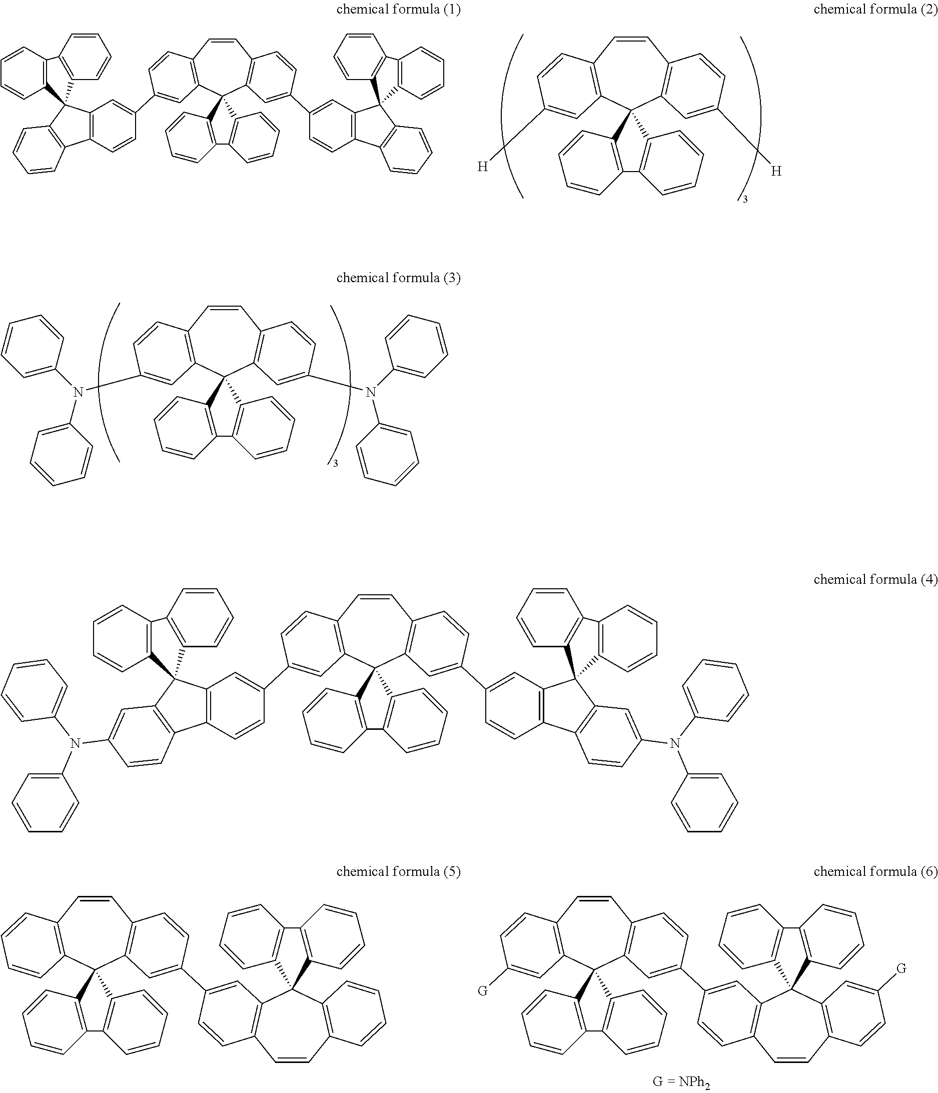

In one embodiment, the spirally configured cis-stilbene/fluorene hybrid material has glass transition temperatures ranged from 221° C. to 231° C.

In one embodiment, the spirally configured cis-stilbene/fluorene hybrid material has decomposition temperatures ranged from 491° C. to 535° C.

In one embodiment, the spirally configured cis-stilbene/fluorene hybrid material has g oxidation potentials ranged from +0.42V to +0.86V and reduction potentials ranged from −2.44V to −2.64V.

In one embodiment, the organic luminescent unit comprises an organic luminescent layer.

In one embodiment, the organic luminescent unit further comprises a hole transport layer and an electron transport layer, and the organic luminescent layer is deposited between the hole transport layer and the electron transport layer.

In one embodiment, the organic luminescent unit further comprises a hole injection layer, a hole transport layer, an electron transport layer and an electron injection layer, and the hole transport layer, the organic luminescent layer and the electron transport layer are sequentially deposited between the hole injection layer and the electron injection layer.

In one embodiment, the organic luminescent layer comprises the spirally configured cis-stilbene/fluorene hybrid material.

In one embodiment, the organic luminescent layer comprises a host material and a guest material, and the host material or the guest material comprises the spirally configured cis-stilbene/fluorene hybrid material.

In one embodiment, the content of the guest material in the organic luminescent layer is between 3 wt % to 15 wt %.

In one embodiment, the content of the guest material in the organic luminescent layer is 15 wt %.

As mentioned above, in the spirally configured cis-stilbene/fluorene hybrid material and the organic electroluminescent device by using the same according to the present invention, it utilizes the spirally configured cis-stilbene/fluorene hybrid or its oligomer, such as dimer or trimer, as the core template. The material according to the present invention has excellent electroluminescent efficiency and high fluorescent quantum yield. Further, such material can be formed as a film by spin-coating, which has a lower cost than that formed by the conventional vacuum evaporation does. Moreover, the material is easily purified so as to prevent the decay of device efficiency and lifespan. In addition, the excellent film ductility of the material takes advantage in flexible device fabrication for organic electroluminescent display and light source.

### BRIEF DESCRIPTION OF THE DRAWINGS

The embodiments will become more fully understood from the detailed description and accompanying drawings, which are given for illustration only, and thus are not limitative of the present invention, and wherein:

FIG. 1 is a cross-sectional schematic diagram of an organic electroluminescent device of the second embodiment according to the invention;

FIG. 2 is a cross-sectional schematic diagram of an organic electroluminescent device of the third embodiment according to the invention; and

FIG. 3 is a cross-sectional schematic diagram of an organic electroluminescent device of the fourth embodiment according to the invention.

### DETAILED DESCRIPTION OF THE INVENTION

The embodiments of the invention will be apparent from the following detailed description, which proceeds with reference to the accompanying drawings, wherein the same references relate to the same elements.

Spirally Configured Cis-Stilbene/Fluorene Hybrid Material

A spirally configured cis-stilbene/fluorene hybrid material according to the first embodiment of the present invention has a structure of the following General Formula (1).

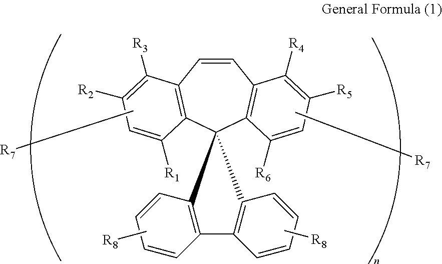

Herein, n is 1, 2 or 3. R₁ to R₆ are each independently selected from the group consisting of a hydrogen atom, halogen atom, cyano group, alkyl group, cycloalkyl group, alkoxy group, amino group, haloalkyl group, thioalkyl group, silyl group, alkenyl group and aromatic ring group. R₈ is a hydrogen atom, tert-butyl group or aromatic ring group.

When n is 1, R₇ is 9,9′-spirobifluorene group, diarylamino group or 2-diarylamino-spirobifluorene group. When n is 2 or 3, R₇ is a hydrogen atom, diarylamino group or 2-diarylamino-spirobifluorene group.

In the present embodiment, the alkyl group can be selected from the group consisting of a substituted or unsubstituted straight-chain alkyl group with the carbon number of 1 to 6, and a substituted or unsubstituted branched-chain alkyl group with the carbon number of 3 to 6. The cycloalkyl group can be a substituted or unsubstituted cycloalkyl group with the carbon number of 3 to 6. The alkoxy group can be selected from the group consisting of a substituted or unsubstituted straight-chain alkoxy group with the carbon number of 1 to 6, and a substituted or unsubstituted branched-chain alkoxy group with the carbon number of 3 to 6. The amino group can be selected from the group consisting of secondary amino group and tertiary amino group. The haloalkyl group can be selected from the group consisting of a substituted or unsubstituted straight-chain haloalkyl group with the carbon number of 1 to 6, and a substituted or unsubstituted branched-chain haloalkyl group with the carbon number of 3 to 6. The thioalkyl group can be selected from the group consisting of a substituted or unsubstituted straight-chain thioalkyl group with the carbon number of 1 to 6, and a substituted or unsubstituted branched-chain thioalkyl group with the carbon number of 3 to 6. The silyl group can be selected from the group consisting of a substituted or unsubstituted straight-chain silyl group with the carbon number of 1 to 6, and a substituted or unsubstituted branched-chain silyl group with the carbon number of 3 to 6. The alkenyl group can be selected from the group consisting of a substituted or unsubstituted straight-chain alkenyl group with the carbon number of 2 to 6, and a substituted or unsubstituted branched-chain alkenyl group with the carbon number of 3 to 6.

Moreover, the secondary amino group can be an amino group having one aromatic ring substituent (for example, a phenyl amino group) or having one C1-C6 straight-chain alkyl, branch-chain alkyl, or non-aromatic ring (for example, a methyl amino group). The tertiary amino group can be an amino group having two independent aromatic ring substituents (for example, a diphenyl amino group, —NPh₂) or having two independent C1-C6 straight-chain alkyl, branch-chain alkyl, or non-aromatic ring (for example, a dimethyl amino group).

The spirally configured cis-stilbene/fluorene hybrid material according to the present embodiment represented by General Formula (1) can be a material of an organic luminescent layer in an organic electroluminescent device, especially can be a guest material. Of course, the material also can be a host material. A preferred example is the compound of chemical formula (1), where n is 1, R₁ to R₆ are all independent hydrogen atoms, R₇ is 9,9′-spirobifluorene group.

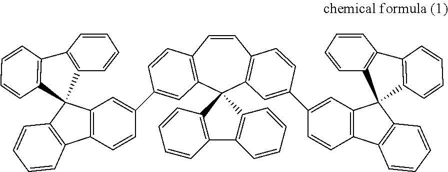

Alternatively, another preferred example is the compound of chemical formula (2), where n is 3, R₁ to R₇ are all independent hydrogen atoms.

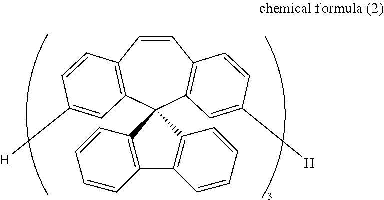

Alternatively, another preferred example is the compound of chemical formula (3), where n is 3, R₁ to R₆ are all independent hydrogen atoms, R₇ is diphenylamino group.

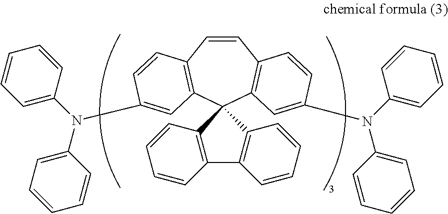

Alternatively, another preferred example is the compound of chemical formula (4), where n is 1, R₁ to R₆ are all independent hydrogen atoms, R₇ is 2-diphenylamino-spirobifluorene group.

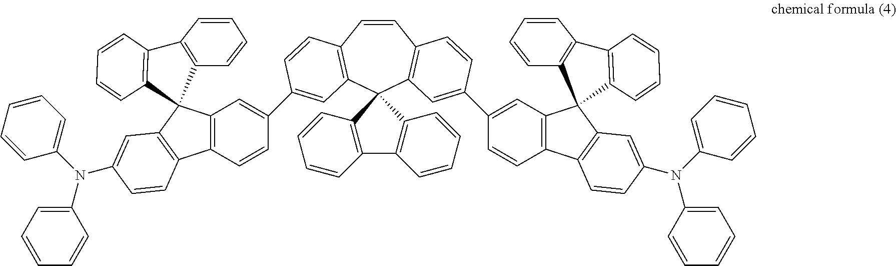

Alternatively, another preferred example is the compound of chemical formula (5), where n is 2, R₁ to R₇ are all independent hydrogen atoms.

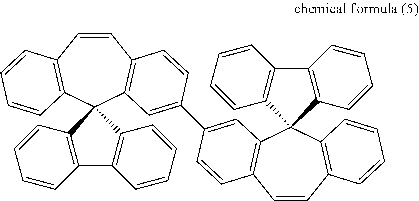

Alternatively, another preferred example is the compound of chemical formula (6), where n is 2, R₁ to R₆ are all independent hydrogen atoms, R₇ is diphenylamino group.

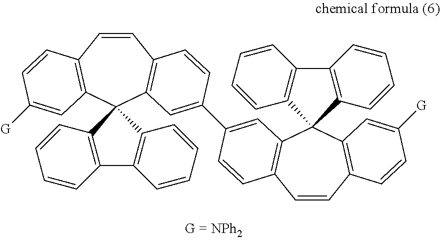

Moreover, in the present embodiment, the spirally configured cis-stilbene/fluorene hybrid materials have glass transition temperatures ranged from 221° C. to 231° C., decomposition temperatures ranged from 491° C. to 535° C., oxidation potentials ranged from +0.42V to +0.86V and reduction potentials ranged from −2.44V to −2.64V.

Organic Electroluminescent Device

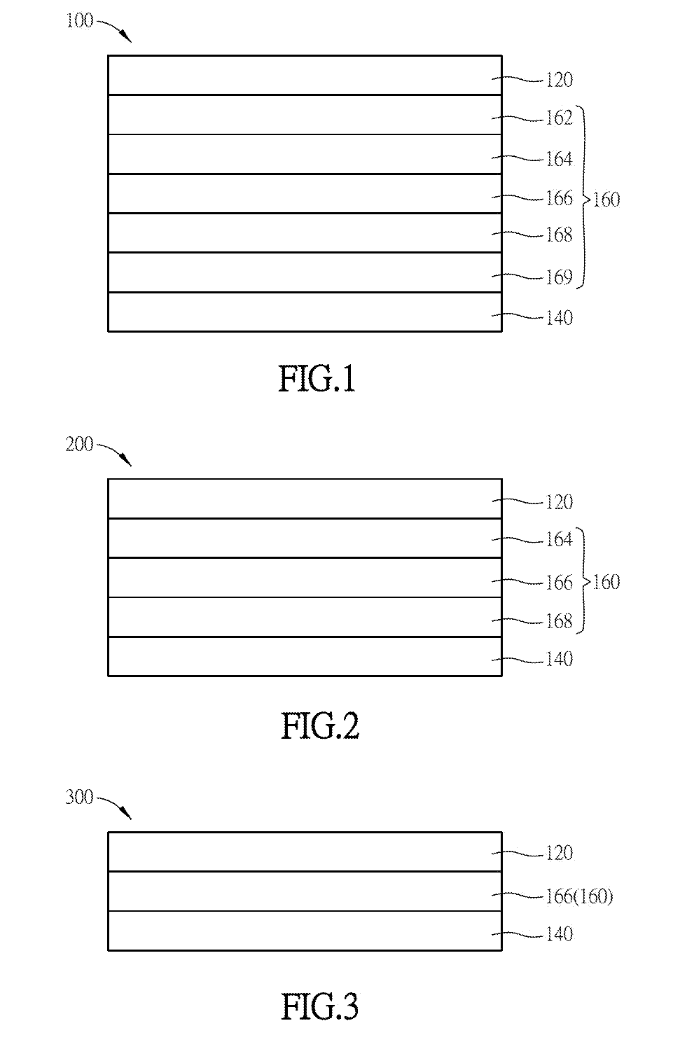

Please refer to FIG. 1, an organic electroluminescent device 100 of the second embodiment according to the invention includes a first electrode layer 120, a second electrode layer 140 and an organic luminescent unit 160. In the embodiment, the first electrode layer 120 can be a transparent electrode material, such as indium tin oxide (ITO), and the second electrode layer 140 can be a metal, transparent conductive substance or any other suitable conductive material. On the other hand, the first electrode layer 120 can also be a metal, transparent conductive substance or any other suitable conductive material, and the second electrode layer 140 can also be a transparent electrode material. Specifically, at least one of the first electrode layer 120 and the second electrode layer 140 of the present embodiment is a transparent electrode material, so that the light emitted from the organic luminescent unit 160 may pass through the transparent electrode, thereby enabling the organic electroluminescent device 100 to emit light.

In addition, please also refer to FIG. 1, the organic luminescent unit 160 can comprise a hole injection layer 162, a hole transport layer 164, an organic luminescent layer 166, an electron transport layer 168 and an electron injection layer 169. The hole transport layer 164, the organic luminescent layer 166 and the electron transport layer 168 are sequentially deposited between the hole injection layer 162 and the electron injection layer 169.

Herein, the materials of the hole injection layer 162 can be poly(3,4-ethylenedioxythiophene) polystyrene sulfonate (PEDOT:PSS). Moreover, the thickness of the hole injection layer 162 of the embodiment is, for example, less than 40 nm. The materials of the hole transport layer 164 can be 1,1-Bis[4-[N,N′-di(p-tolyl)amino]phenyl]cyclohexane (TAPC), N,N-bis-(1-naphthyl)-N,N-diphenyl-1,1-biphenyl-4,4-diamine (NPB) or N—N′-diphenyl-N—N′bis(3-methylphenyl)-[1-1′-biphenyl]-4-4′-diamine (TPD) and so on. In the embodiment, the hole injection layer 162 and the hole transport layer 164 can increase the injection rate of hole transported from the first electrode layer 120 to the organic luminescent layer 166 and can also reduce the driving voltage of the organic electroluminescent device 100.

The materials of the electron transport layer 168 can be 2,2,2-(1,3,5-benzinetriyl)-tris(1-phenyl-1-H-benzimidazole) (TBPI) or 1,3-Bis[3,5-di(pyridin-3-yl)phenyl]benzene (BmPyPb). In the embodiment, the thickness of the electron transport layer 168 is, for example, less than 50 nm. In the present embodiment, the electron transport layer 168 may further increase the transport rate of the electron from the electron injection layer 169 to the organic luminescent layer 166. In addition, the materials of the electron injection layer 169 can be LiF and the thickness of the electron injection layer 169 can be between 0.1 nm and 5 nm, for example, 1 nm.

In addition, the thickness of the organic luminescent layer 166 can be between 5 nm and 50 nm, for example, 40 nm. The organic luminescent layer 166 may include the host material and the guest material, and the doping concentration of the guest material (weight percentage) can be ranged from 3 wt % to 20 wt %, for example, 15 wt %.

The host materials can be 4,4′-Bis(N-carbazolyl)-1,1′-biphenyl (CBP), N,N′-Di(1-naphthyl)-N,N′-diphenyl-(1,1′-biphenyl)-4,4′-diamine (NBP), 3,5-di(9H-carbazol-9-yl)tetraphenylsilane (SimCP2), 4,4′,4″-tri(N-carbazolyl)triphenylamine (TCTA), 2,7-bis(carbazo-9-yl)-9,9-ditolyfluorene (Spiro-2CBP) or the host materials with 9, 10-diaryl substituents, for example, 1-butyl-9,10-naphthalene-anthracene (BANE).

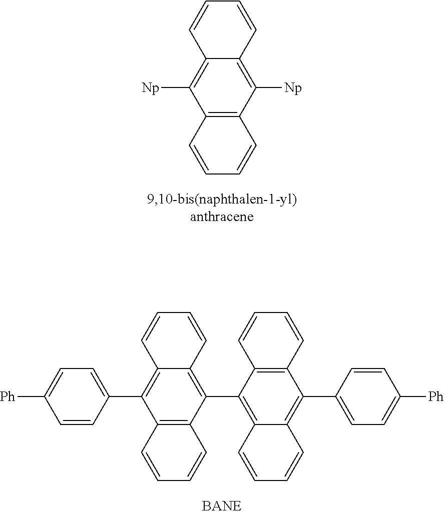

Herein, the guest material can be the spirally configured cis-stilbene/fluorene hybrid material which has a structure of General Formula (1).

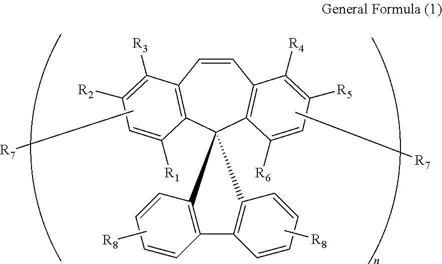

Herein, n is 1, 2 or 3. R₁ to R₆ are each independently selected from the group consisting of a hydrogen atom, halogen atom, cyano group, alkyl group, cycloalkyl group, alkoxy group, amino group, haloalkyl group, thioalkyl group, silyl group, alkenyl group and aromatic ring group. R₈ is a hydrogen atom, tert-butyl group or aromatic ring group.

When n is 1, R₇ is 9,9′-spirobifluorene group, diarylamino group or 2-diarylamino-spirobifluorene group. When n is 2 or 3, R₇ is a hydrogen atom, diarylamino group or 2-diarylamino-spirobifluorene group.

In addition, FIG. 2 is a cross-sectional schematic diagram of an organic electroluminescent device 200 of the third embodiment according to the invention. The configuration of the organic electroluminescent device 200 is substantially similar with that of the organic electroluminescent device 100, and same elements have substantial the same characteristics and functions. Therefore, the similar references relate to the similar elements, and detailed explanation is omitted hereinafter.

Please refer to FIG. 2, in the embodiment, the organic luminescent unit 160 can comprise a hole transport layer 164, an organic luminescent layer 166 and an electron transport layer 168. The organic luminescent layer 166 is deposited between the hole transport layer 164 and the electron transport layer 168.

In addition, FIG. 3 is a cross-sectional schematic diagram of an organic electroluminescent device 300 of the fourth embodiment according to the invention. The configuration of the organic electroluminescent device 300 is substantially similar with that of the organic electroluminescent device 100, and same elements have substantial the same characteristics and functions. Therefore, the similar references relate to the similar elements, and detailed explanation is omitted hereinafter.

Please refer to FIG. 3, in the embodiment, the organic luminescent unit 160 can comprise an organic luminescent layer 166.

Moreover, the configuration of the organic electroluminescent device according to the invention is not limited to what is disclosed in the second, third or fourth embodiment. The second, third and fourth embodiments are embodiments for illustration.

In addition, the various examples, the number of n and the selection of the substituents of R₁ to R₈ in General Formula (1) of the spirally configured cis-stilbene/fluorene hybrid materials in the second, third and fourth embodiments, as well as their properties, such as glass transition temperatures, decomposition temperatures, oxidation potentials and redox potentials, are substantially the same as those in the first embodiment and are therefore omitted here.

To illustrate the synthesis of the compounds represented by chemical formula (1), formula (2) and chemical formula (3), i.e., compounds 9 to 11, there are several synthetic approaches and examples shown below.

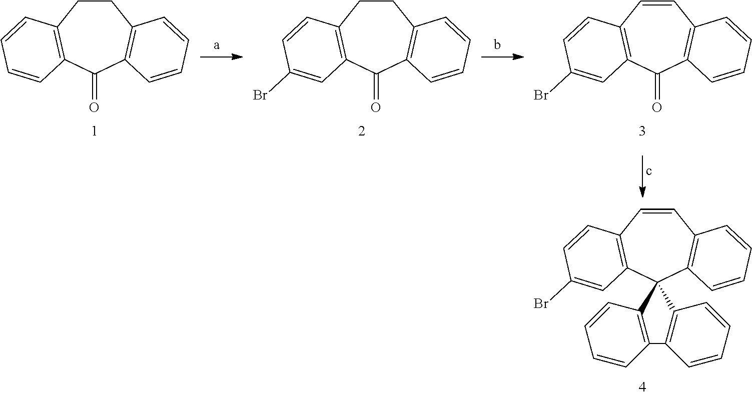

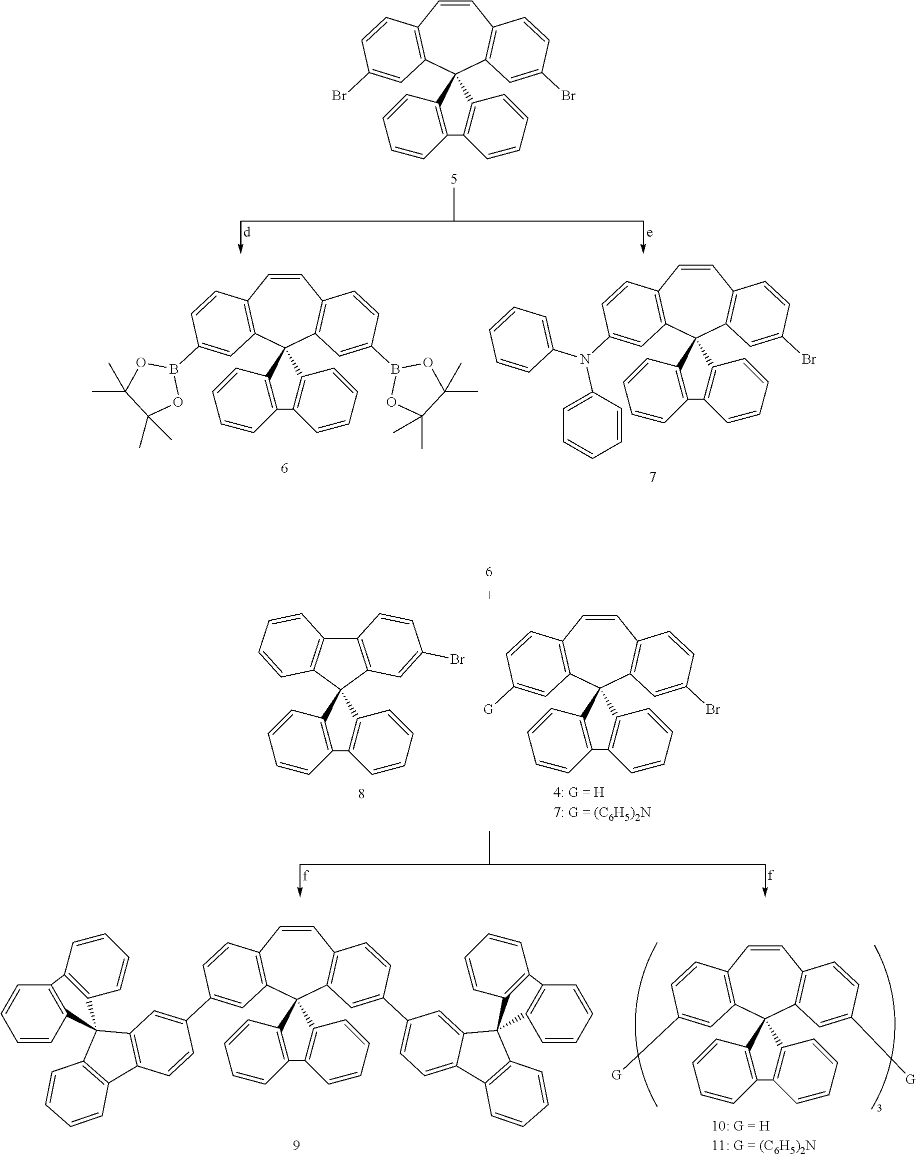

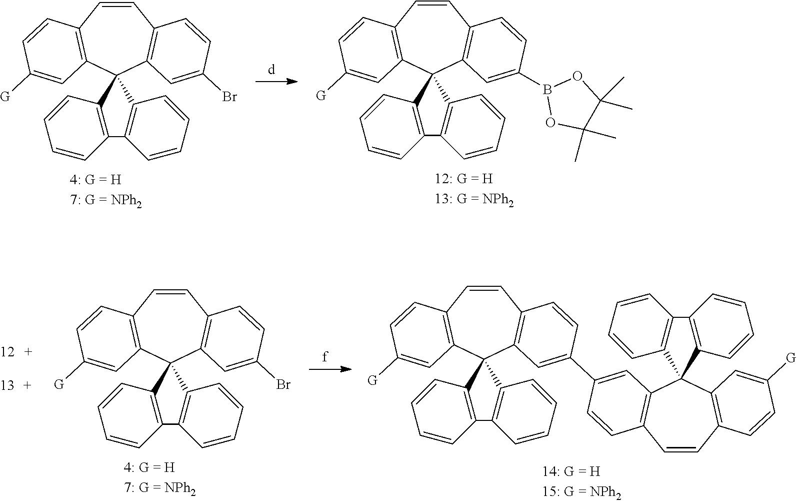

Reagents and conditions: ^{a} AlCl₃, Br₂, 0° C., 3 minutes; ^{b} PCl₅, POCl₃, 100 □, 5 hours; ^{c} n-BuLi and 2-bromobiphenyl, THF, −78° C., 2 hours; ^{d} n-BuLi and 2-isopropoxy-4,4,5,5-tetramethyl-1,3,2-dioxaborolane, THF, −78° C., 2 hours; ^{e} Pd₂(dba)₃, NaOt-Bu, N,N′-diphenylamine and 1,1′-Bis(diphenylphosphino)ferrocene, toluene, reflux, 4 hours; ^{f} Pd(PPh₃)₄ and Na₂CO₃, DME, reflux, 18 hours.

### Example 1: Synthesis of Compound 5 (3,7-dibromo-5,5-spirofluorenyl-5H-dibenzo[a,d]cycloheptene)

An organolithium reagent is prepared by adding 2-bromobiphenyl and n-butyllithium in tetrahydrofuran at −78° C. Then dibromo-substituted dibenzosuberenone dissolved in tetrahydrofuran (3M) was added drop by drop into the organolithium reagent (1.5 N). The crude tertiary alcohol obtained by extracting from quenched reaction mixture was added 0.6 N HOAc/HCl and the mixture refluxed for 0.5 hour (Friedel-Crafts reaction) to proceed the intramolecular cyclization reaction. Followed by recrystallized from dichloromethane, compound 5 was obtained.

### Example 2: Synthesis of Compound 6 (3,7-Bis[(pinacolato)boryl]-5,5-spirofluorenyl-5H-dibenzo[a,d]cycloheptene)

To a 25 mL, three-necked, round-bottomed flask was placed 3,7-dibromo-5,5-spirofluorenyl-5H-dibenzo[a,d]cycloheptene (compound 5) (500 mg, 1 mmol) in THF (10 mL). The reaction flask was cooled to −78° C. and n-BuLi (2.5 M in hexane, 1 mL, 2.5 mmol) was added dropwise. The whole solution was stirred at this temperature for 30 minutes followed by adding 2-isoproxy-4,4,5,5-tetramethyl-1,3,2-dioxaborolane (0.51 mL, 2.5 mmol) under argon atmosphere. The resulting mixture was gradually warmed to ambient temperature and quenched by H₂O (5 mL). The mixture was extracted with CH₂Cl₂ (3×20 mL). The combined organic layers were dried (MgSO₄), filtered, and evaporated under reduced pressure. The crude residue was purified by recrystallized from CH₂Cl₂/hexanes to afford pure compound 6 (327 mg, 55%).

Spectral data as follow: Tₘ 207° C. (DSC); M.W.: 594.35; ¹H NMR (400 MHz, CDCl₃) δ 7.96 (d, J=7.6, 2H), 7.72 (d, J=7.5, 2H), 7.58 (d, J=7.5, 2H), 7.37 (s, 2H), 7.34 (t, J=8.2, 4H), 7.22 (t, J=7.5, 2H), 7.00 (s, 2H), 1.19 (s, 12H); ¹³C NMR (100 MHz, CDCl₃) δ 152.5, 141.3, 139.2, 138.8, 135.4, 134.3, 133.3, 131.4, 127.7, 127.2, 127.1, 120.2, 83.5, 66.0, 24.8; MS (EI, 20 eV) 594.3 (M⁺, 68); TLC R_{f} 0.35 (Acetone/hexanes, 1/1); HR-MS calcd for C₃₉H₄₀B₂O₄: 594.3113, found: 594.3120.

### Example 3: Synthesis of Compound 9 (Chemical Formula (1), 3,7-Bis(2-spirobifluorene-yl)-5,5-spirofluorenyl-5H-dibenzo[a,d]cycloheptene)

To a 50 mL, two-necked, round-bottomed flask was placed compound 6 (594 mg, 1 mmol) and Pd(PPh₃)₄ (35 mg, 0.03 mmol) in dimethoxyethane (25 mL). A solution of 2-bromo-spirobifluorene (commercially available compound 8, 949 mg, 2.4 mmol) and Na₂CO₃ (318 mg, 3 mmol) in degassed water (12 mL) was added and the resulting mixture was refluxed for 24 hours. The reaction mixture was cooled to ambient temperature and quenched with water (20 mL). The whole mixture was extracted with dichloromethane (3×20 mL). The combined organic layers were dried (MgSO₄), filtered, and evaporated. The crude residue was purified by column chromatography on silica gel to give compound 9, which was further recrystallized from CH₂Cl₂ to afford pure compound 9 (675 mg, 61%).

Spectral data as follow: Tₘ 427° C. (DSC); T_{d} 509° C. (TGA); T_{g} 226° C. (DSC); M.W.: 971.19; ¹H NMR (400 MHz, CDCl₃) δ 7.95 (d, J=7.6, 4H), 7.79 (t, J=7.4, 4H), 7.74 (d, J=7.9, 2H), 7.50 (d, J=7.6, 2H), 7.45 (td, J=7.8, 0.6, 4H), 7.35 (td, J=7.8, 0.6, 2H), 7.29-7.25 (m, 6H), 7.15-7.07 (m, 8H), 6.97 (s, 2H), 6.91-6.87 (m, 4H), 6.71 (d, J=7.6, 2H), 6.68 (d, J=7.6, 4H), 6.57 (d, J=1.2, 2H); ¹³C NMR (100 MHz, CDCl₃) δ 152.2, 149.4, 149.1, 148.7, 141.8, 141.7, 141.4, 141.0, 140.3, 140.0, 138.8, 135.4, 132.7, 132.5, 128.0, 127.9, 127.8, 127.8, 127.7, 127.0, 126.8, 126.1, 125.2, 124.2, 124.0, 122.3, 120.1, 120.0, 119.9, 66.0, 65.9; MS (FAB) 970.4 (M⁺, 76); TLC R_{f} 0.35 (CH₂Cl₂/hexanes, 1/3); HR-MS calcd for C₇₇H₄₆: 970.3600, found: 970.3607; Anal. Calcd for C₇₇H₄₆: C, 95.23, H, 4.77. Found: C, 95.03, H, 4.76.

### Example 4: Synthesis of Compound 2 (3-Bromo-10,11-dihydro-dibenzo[a,d]cyclohepten-5-one)

To a 50 mL, two-necked, round-bottomed flask was placed AlCl₃ (1467 mg, 11 mmol) and Br₂ (1.5 mL, 30 mmol) and stirred for 15 minutes at 0° C. Dibenzosuberone (1041 mg, 5 mmol) was added slowly and the whole mixture was stirred for another 3 minutes under argon atmosphere. The resulting mixture was gradually warmed to ambient temperature and quenched by ice water (10 mL). The mixture was extracted with EtOAc (3×15 mL). The combined organic layers were dried (MgSO₄), filtered, and evaporated under reduced pressure. The crude residue was purified by column chromatography on silica gel to give compound 2 (517 mg, 30%).

Spectral data as follow: Tₘ 80° C. (DSC); M.W.: 287.15; ¹H NMR (400 MHz, CDCl₃) δ 8.14 (d, J=2, 1H), 7.99 (d, J=7.8, 1H), 7.53 (dd, J=8.1, 2, 1H), 7.45 (t, J=7.4, 1H), 7.34 (t, J=7.6, 1H), 7.23 (d, J=7.5, 1H), 7.11 (d, J=8.1, 1H), 3.18 (m, 4H); ¹³C NMR (100 MHz, CDCl3) δ 193.9, 141.8, 140.8, 140.1, 138.0, 135.0, 133.3, 132.7, 131.1, 130.7, 129.3, 126.8, 120.5, 34.7, 34.4; MS (EI, 20 eV) 286.1 (M⁺, 94); TLC R_{f} 0.60 (EtOAc/hexanes, 1/20).

### Example 5: Synthesis of Compound 3 (3-Bromo-dibenzo[a,d]cyclohepten-5-one)

To a 50 mL, two-necked, round-bottomed flask was placed a solution of compound 2 (2872 mg, 10 mmol) and phosphorous pentachloride (4581 mg, 22 mmol) in POCl₃ (9 mL). The whole solution was heated to 90° C. for 4 hours followed by direct hydrolysis. The reaction mixture was added into a mixture CH₂Cl₂ (20 mL), MeOH (10 mL) and ice water (10 mL) slowly and stirred for 12 hours. The mixture was extracted with EtOAc (3×20 mL). The combined organic layers were dried (MgSO₄), filtered, and evaporated under reduced pressure. The crude residue was purified by column chromatography on silica gel to give compound 3, which was further recrystallized from EtOH to afford pure compound 3 (2423 mg, 85%).

Spectral data as follow: Tₘ 108° C. (DSC); M.W.: 285.14; ¹H NMR (400 MHz, CDCl₃) δ 8.37 (d, J=2.1, 1H), 8.22 (d, J=7.9, 1H), 7.77 (dd, J=8.3, 2.1, 1H), 7.67 (t, J=7.4, 1H), 7.57 (t, J=7.7, 2H), 7.42 (d, J=8.3, 1H), 7.10 (d, J=12.1, 1H), 7.01 (d, J=12.1, 1H); ¹³C NMR (100 MHz, CDCl₃) δ 191.4, 139.8, 138.3, 134.9, 133.7, 132.9, 132.4, 132.3, 132.3, 131.0, 131.6, 130.4, 129.1, 123.2; MS (EI, 20 eV) 284.1 (M⁺, 38); TLC R_{f} 0.35 (EtOAc/hexanes, 1/20).

### Example 6: Synthesis of Compound 4 (3,-Bromo-5,5-spirofluorenyl-5H-dibenzo[a,d]cycloheptene)

To a 250 mL, three-necked, round-bottomed flask was placed a solution of 2-bromobiphenyl (2331 mg, 10 mmol) in THF (50 mL). The reaction flask was cooled to −78° C. and n-BuLi (2.5 M in hexane, 4 mL, 10 mmol) was added dropwise. The whole solution was stirred at this temperature for 30 minutes followed by dropwisely adding a solution of compound 3 (2851 mg, 10 mmol) in THF (30 mL) under argon atmosphere. The resulting mixture was gradually warmed to ambient temperature and quenched by saturated, aqueous NaHCO₃ (30 mL). The mixture was extracted with CH₂Cl₂ (3×50 mL). The combined organic layers were dried (MgSO₄), filtered, and evaporated under reduced pressure. The crude residue was placed in another 100 mL, two-necked, round-bottomed flask and dissolved in acetic acid (15 mL). Catalytic amount of aqueous HCl (5 mol %, 12N) was then added and the whole solution was refluxed for 15 minutes. After having been cooled to ambient temperature, the whole mixture was extracted with dichloromethane (3×20 mL). The combined organic layers were dried (MgSO₄), filtered, and evaporated under reduced pressure. The crude residue was purified by column chromatography on silica gel to give compound 4, which was further recrystallized from CH₂Cl₂/hexanes to afford pure compound 4 (3834 mg, 91%).

Spectral data as follow: Tₘ 231° C. (DSC); M.W.: 421.33; ¹H NMR (400 MHz, CDCl3) δ 7.94 (d, J=7.8, 2H), 7.75 (d, J=7.6, 2H), 7.41-7.17 (m, 8H), 7.03 (d, J=1.5, 1H), 6.99-6.92 (m, 2H), 6.90-6.86 (m, 2H); ¹³C NMR (100 MHz, CDCl3) δ 152.2, 143.8, 141.5, 138.9, 136.2, 135.4, 133.9, 133.4, 132.3, 132.0, 131.8, 130.3, 128.9, 128.7, 128.2, 127.7, 127.5, 126.9, 122.5, 120.4, 65.7; MS (EI, 20 eV) 420.0 (M+, 100); TLC R_{f} 0.35 (CH₂Cl₂/hexanes, 1/5); HR-MS calcd for C₂₇HᵣBr: 420.0514, found: 420.0494.

### Example 7: Synthesis of Compound 10 (Chemical Formula (2), 3,7-Bis(3-spirodibenzosuberenefluorene-yl)-5,5-spirofluorenyl-5H-dibenzo[a,d]cycloheptene)

To a 50 mL, two-necked, round-bottomed flask was placed compound 6 (594 mg, 1 mmol) and Pd(PPh₃)₄ (35 mg, 0.03 mmol) in DME (25 mL). A solution of compound 4 (1011 mg, 2.4 mmol) and Na₂CO₃ (318 mg, 3 mmol) in degassed water (12 mL) was added and the resulting mixture was refluxed for 24 hours. The reaction mixture was cooled to ambient temperature and quenched with water (20 mL). The whole mixture was extracted with dichloromethane (3×20 mL). The combined organic layers were dried (MgSO₄), filtered, and evaporated. The crude residue was purified by column chromatography on silica gel to give compound 10 (758 mg, 78%).

Spectral data as follow: Tₘ 497° C. (TGA); T_{g} 231° C. (DSC); M.W.: 1023.26; ¹H NMR (400 MHz, CDCl₃) δ 7.89 (d, J=7.7, 4H), 7.84 (d, J=7.7, 2H), 7.67 (d, J=7.9, 4H), 7.64 (d, J=8.0, 2H), 7.34-7.14 (m, 20H), 6.94-6.85 (m, 18H); ¹³C NMR (100 MHz, CDCl₃) δ 152.7, 152.4, 142.0, 141.8, 141.6, 140.3, 140.3, 138.9, 138.8, 136.4, 135.4, 133.4, 132.8, 132.7, 132.7, 132.6, 132.2, 128.8, 128.4, 127.9, 127.9, 127.6, 127.5, 127.4, 127.4, 127.2, 126.9, 126.8, 125.4, 120.3, 120.3, 66.0; MS (FAB) 1022.4 (M⁺, 96); TLC R_{f} 0.35 (CH₂Cl₂/hexanes, 1/4); HR-MS calcd for C₈₁H₅₀: 1022.3913, found: 1022.3922.

### Example 8: Synthesis of Compound 7 (3-bormo-7-(N,N-diphenylamino)-5,5-spirofluorenyl-5H-dibenzo[a,d]cycloheptene)

To a 50 mL, two-necked, round-bottomed flask was placed compound 5 (500 mg, 1 mmol), Pd₂(dba)₃ (23 mg, 0.02 mmol), sodium tert-butoxide (288 mg, 3 mmol), 1,1′-bis(diphenylphosphino)ferrocene (18 mg, 0.03 mmol) and diphenylamine (169 mg, 1 mmol) in toluene (25 mL). The whole solution was refluxed for 12 hours. The reaction mixture was quenched with saturated aqueous NaHCO₃ (20 mL) and extracted with CH₂Cl₂ (3×20 mL). The combined organic layers were dried (MgSO₄), filtered, and evaporated. The crude residue was purified by column chromatography on silica gel to give compound 7, which was further re-crystallized from CH₂Cl₂/hexanes to afford pure compound 7 (282 mg, 48%).

Spectral data as follow: Tₘ 256° C. (DSC); M.W.: 588.53; ¹H NMR (400 MHz, CDCl3) δ 7.78 (d, J=7.8, 2H), 7.62 (d, J=7.5, 2H), 7.29-7.23 (m, 3H), 7.19-7.14 (m, 6H), 7.02-6.99 (m, 5H), 6.91-6.88 (m, 6H), 6.77 (d, J=12.1, 1H), 6.52 (d, J=2.2, 1H); ¹³C NMR (100 MHz, CDCl3) δ 151.7, 148.6, 146.9, 138.7, 135.8, 133.5, 133.2, 133.1, 131.6, 130.1, 129.7, 129.7, 129.2, 127.9, 127.6, 126.9, 125.0, 123.4, 120.1, 119.9, 65.6; MS (MALDI-TOF) 588.0 (M⁺, 56); TLC R_{f} 0.45 (CH₂Cl₂/hexanes, 1/3); HR-MS calcd for C₃₉H₂₆BrN: 587.1249, found: 587.9560.

### Example 9: synthesis of compound 11 (chemical formula (3), 3,7-Bis[3-(7-diphenylamino)spirodibenzosuberenefluorene-yl]-5,5-spirofluor-enyl-5H-dibenzo[a,d]cyclo-heptene)

To a 50 mL, two-necked, round-bottomed flask was placed compound 6 (594 mg, 1 mmol) and Pd(PPh₃)₄ (35 mg, 0.03 mmol) in DME (25 mL). A solution of compound 7 (1412 mg, 2.4 mmol) and Na₂CO₃ (318 mg, 3 mmol) in degassed water (12 mL) was added and the resulting mixture was refluxed for 24 hours. The reaction mixture was cooled to ambient temperature and quenched with water (20 mL). The whole mixture was extracted with dichloromethane (3×20 mL). The combined organic layers were dried (MgSO₄), filtered, and evaporated. The crude residue was purified by column chromatography on silica gel to give compound 11 (625 mg, 46%).

Spectral data as follow: T_{d} 535° C. (TGA); T_{g} 231° C. (DSC); M.W.: 1359.68; ¹H NMR (400 MHz, CDCl₃) δ 7.82 (d, J=7.7, 2H), 7.74 (d, J=7.8, 4H), 7.62 (d, J=7.9, 2H), 7.55 (d, J=7.4, 4H), 7.29-7.13 (m, 24H), 7.00-6.93 (m, 8H), 6.89-6.78 (m, 24H), 6.54 (d, J=2.1, 2H); ¹³C NMR (100 MHz, CDCl₃) δ 152.4, 152.0, 148.3, 147.0, 141.9, 141.7, 141.1, 140.5, 139.9, 138.8, 138.7, 135.8, 135.3, 133.0, 132.7, 132.6, 132.4, 130.5, 130.2, 129.1, 127.9, 127.6, 127.4, 127.3, 127.0, 126.8, 125.4, 124.9, 123.7, 123.2, 120.3, 120.1, 120.0, 65.9; MS (MALDI-TOF) 1358.8 (M+H⁺, 100); TLC R_{f} 0.25 (CH₂Cl₂/hexanes, 2/9); HR-MS calcd for C₁₀₅H₆₉N₂: 1358.5494, found: 1358.7750.

### Example 10: Synthesis of Compound 15 (Chemical Formula (6), N7,N7,N7″,N7″-tetraphenyl-3,3″-bispiro[dibenzo[a,d][7]annulene-5,9′-fluorene]-7,7″-diamine

To a 25 mL, three-necked, round-bottomed flask was placed compound 7 (500 mg, 1 mmol) in THF (10 mL). The reaction flask was cooled to −78° C. and n-BuLi (2.5 M in hexane, 0.6 mL, 1.5 mmol) was added dropwise. The whole solution was stirred at this temperature for 30 minutes followed by adding 2-isoproxy-4,4,5,5-tetramethyl-1,3,2-dioxaborolane (0.26 mL, 1.3 mmol) under argon atmosphere. The resulting mixture was gradually warmed to ambient temperature and quenched by H₂O (5 mL). The mixture was extracted with CH₂Cl₂ (3×20 mL). The combined organic layers were dried (MgSO₄), filtered, and evaporated under reduced pressure and crude residue 13 (381.4 mg, 60%) was obtained. To a 50 mL, two-necked, round-bottomed flask was placed compound 13 (320 mg, 0.5 mmol) and Pd(PPh₃)₄ (18 mg, 0.015 mmol) in DME (15 mL). A solution of compound 7 (353.6 mg, 0.6 mmol) and Na₂CO₃ (165 mg, 1.5 mmol) in degassed water (6 mL) was added and the resulting mixture was refluxed for 18 hours. The reaction mixture was cooled to ambient temperature and quenched with water (12 mL). The whole mixture was extracted with dichloromethane (3×15 mL). The combined organic layers were dried (MgSO₄), filtered, and evaporated. The crude residue was purified by column chromatography on silica gel to give compound 15 (chemical formula (6), 742 mg, 73%).

Spectral data as follow: T_{d} 512° C. (TGA); T_{g} 221° C. (DSC); M.W.: 1017.29; ¹H NMR (400 MHz, CDCl₃) δ 7.92 (dd, J=8.0, 1.8, 4H), 7.53 (d, J=2.0, 2H), 7.62 (d, J=8.0, 2H), 7.55 (d, J=1.8, 4H), 7.53 (dd, J=7.8, 1.9, 2H), 7.42-7.37 (m, 6H), 7.29-7.23 (m, 6H), 7.09-7.01 (m, 12H), 6.98 (s, 4H); ¹³C NMR (100 MHz, CDCl₃) δ 152.4, 152.0, 148.3, 147.0, 141.9, 141.7, 141.1, 140.5, 139.9, 146.1, 141.9, 141.6, 141.1, 140.5, 134.3, 129.8, 129.6, 129.1, 128.8, 128.1, 127.8, 127.4, 126.8, 126.7, 126.5, 125.8, 125.2, 122.4, 121.5, 61.6; MS (MALDI-TOF) 1018.3 (M+H⁺, 100); TLC R_{f}0.21 (CH₂Cl₂/hexanes, 2/9); HR-MS calcd for C₇₈H₅₂N₂: 1016.4130, found: 1016.4127.

Measurement Data (Thermal Stabilities, Photophysical Properties and Electrochemical Properties) of the Compounds of Chemical Formula (1), Chemical Formula (2), Chemical Formula (3) and Chemical Formula (6) are Shown in Table 1.

TABLE 1
(obtained by steady-state measurement):

| | | Abs. | Em. | | Eₒₓ^{[a]}/ |
|---|---|---|---|---|---|
| | T_{g}/T_{d} | λₘₐₓ^{[a,b]} | λₘₐₓ^{[a,c]} | Φ_{f} | E_{red}^{[d]} |
| Compound | (° C.) | (nm) | (nm) | (%) | (V) |
| Chemical | 226/509 | 276 (43.3) | 434, | 99 | +0.86/ |
| formula (1) | | 310 (38.8) | 459 (61) | | −2.44, |
| | | 376 (32.8) | | | −2.64 |
| Chemical | 231/491 | 306 (44.9) | 444, | 99 | N.A./ |
| formula (2) | | 370 (34.7) | 462 (61) | | −2.53 |
| Chemical | 231/535 | 264 (78.7) | 499, | 85 | +0.42/ |
| formula (3) | | 295 (56.0) | 522 (87) | | −2.46 |
| | | 415 (66.8) | | | |
| Chemical | 221/512 | 306 (25.2) | 454(62) | 99 | +0.46/ |
| formula (6) | | 351 (10.6) | | | −2.50 |
| | | 400 (25.8) | | | |
| Compound 12 | N.A./450 | 264 (61.9) | 394, | 99 | +1.32, |
| ter(9,9- | | 310 (50.1) | 415 (44) | | +1.56/ |
| spirobifluorene) | | 351 (90.8) | | | −2.01, |
| | | | | | −2.21 |

^{[a]}Measured in CH₂Cl₂.
^{[b]}The data in parentheses correspond to ε x 10⁻³.
^{[c]}The data in parentheses correspond to full width at half-maximum.
^{[d]}Measured in THF.

Regarding to the thermal stabilities, the decomposition temperature (T_{d}) of the compound is measured by thermogravimetric analyzer (TGA), and the glass transition temperature (T_{g}) of the compound is measured by differential scanning calorimeter (DSC).

According to Table 1, the decomposition temperatures of chemical formula (1), chemical formula (2), chemical formula (3) and chemical formula (6) are all higher than 490° C., in particular, chemical formula (3) has the highest decomposition temperature of 535° C. In addition, the glass transition temperatures of chemical formula (1), chemical formula (2), chemical formula (3) and chemical formula (6) are all higher than 220° C. Herein, the two spirobifluorene units of chemical formula (1) are twisted by 31.5 and 42.3°, respectively, to central spiro[dibenzosuberene-fluorene] template so that chemical formula (1) has excellent thermal stabilities. On the other hand, each fluorene damper is nearly perpendicular)(89±0.3° to the top moiety for all of chemical formula (1), chemical formula (2), chemical formula (3) and chemical formula (6). Such spirally shaped structure may contribute to the increase in glass transition temperature (T_{g}, 221-231° C.) and thermal decomposition temperature (T_{d}, 491-535° C.). Overall, all of chemical formula (1), chemical formula (2), chemical formula (3) and chemical formula (6) are materials with high thermal stabilities.

Moreover, the steady-state photophysical properties of the materials in Table 1 were obtained under atmosphere at ambient temperature. There are longest absorption bands with peak maxima (λₘₐₓ) appeared at 376, 370, 415 and 400 nm, for chemical formula (1), chemical formula (2), chemical formula (3) and chemical formula (6), respectively. Notably, a dramatic bathochromic shift (42±3 nm) was observed for chemical formula (3). That is because the electron densities are completely delocalized to the whole oligomeric chains at the HOMOs for all of chemical formula (1), chemical formula (2), chemical formula (3) and chemical formula (6). However, chemical formula (3) and chemical formula (6) were end-capped with diphenylamino groups. The strong electron-donating ability attributes to more localized electrons toward the central ter-cis-stilbene moiety at the corresponding LUMO, and consequently reduces its lowest-lying transition energy (n=0→1). In addition, the redox behaviours of chemical formula (1), chemical formula (2), chemical formula (3) and chemical formula (6) were evaluated by cyclic voltammetry experiments using ferrocene as an internal reference. The redox couples were observed at +0.86, +0.42 and +0.46 V for chemical formula (1), chemical formula (3) and chemical formula (6), respectively, which refer to the oxidation of spirobifluorene and diphenylamine units, whereas no apparent oxidation potential (Eₒₓ) was observed below +1.5 V for chemical formula (2). Contrarily, all of chemical formula (1), chemical formula (2), chemical formula (3) and chemical formula (6) have similar reduction potentials (E_{red}). In addition, the redox couples were observed at −2.49±0.05 V for chemical formula (1), chemical formula (2), chemical formula (3) and chemical formula (6) correspond to the reduction of each stilbene unit, while another one observed at −2.64 V for chemical formula (1) implies the reduction of fluorene fragment at the oligo-trimeric chain.

TABLE 2
(time resolved transient spectra obtained during the pulse radiolysis):

| | Abs. | Abs. | Abs. | Em. λₘₐₓ^{[d,e]}/ |
|---|---|---|---|---|
| | λₘₐₓ^{[a,b]} | λₘₐₓ^{[b,c]} | λₘₐₓ^{[d]} | Int.^{[d]} |
| Compound | (nm) | (nm) | (nm) | (nm/%) |
| Chemical formula (1) | 620 (3.9) | 670 (7.1) | 650 | 463 (63)/60 |
| Chemical formula (2) | 620 (3.5) | 685 (13.3) | 700 | 473 (61)/64 |
| Chemical formula (3) | 570 (11.3) | 690 (14.2) | 655 | 521 (89)/109 |
| Compound 12 | 550 (7.1) | 570 (5.1) | 667 | 425 (32)/100 |
| ter(9,9-spirobifluorene) | | | | |

^{[a]}Measured in dichloroethene (DCE).
^{[b]}The data in parentheses correspond to half-lives (μs).
^{[c]}Measured in dimethylformamide (DMF).
^{[d]}Measured in benzene (the total amount of emission relative to that of ter(9,9-spirobifluorene) is 100%).
^{[e]}The data in parentheses correspond to full width at half-maximum.

The similar transient absorption spectra (λₘₐₓ at 620 nm) and short τ_{1/2} values (3.7±0.2 μs) observed in DCE solution for chemical formula (1) and chemical formula (2) imply that their radical cations are delocalized in the whole oligo-trimeric chains. Oppositely, a hypsochromic shifted absorption with λₘₐₓ at 570 nm (τ_{1/2}=11.3 μs) was observed for chemical formula (3). In comparison with that for the monomer having the same diphenylamino substituents at the C3 and C7 positions, it is assigned to be radical cations localized in the diphenylamine segment. Since the τ_{1/2} value of radical ion obtained during the pulse radiolysis for dipolar molecule has already been proven to have well correlation with the capability of charge transport in device when its energy level matches to the work function of the electrolyte, chemical formula (3) can be reasonably expected to have a good hole transporting function.

In DMF, the transient absorption spectra obtained during the pulse radiolysis for chemical formula (1), chemical formula (2) and chemical formula (3) (λₘₐₓ at 680±15 nm) are similar to each other in their profiles because all the radical anions are resided in the oligo-trimeric chains. According to the trend for their t_{1/2} values (7.1, 13.3, 14.2 for chemical formula (1), chemical formula (2) and chemical formula (3), respectively), the radical anions of chemical formula (2) and chemical formula (3) tend to be stabilized by accelerating neutralization process via lager collision cross section.

The relative emission intensities obtained for chemical formula (1), chemical formula (2) and chemical formula (3), for example, are 60-109% to the reference molecule [ter(9,9-spirobifluorene), compound 12] (100%). In general, chemical formula (3) is suggested to be 1.6 times better than chemical formula (1) and chemical formula (2) in their electroluminescent efficiencies. From these results, the devices using chemical formula (1), chemical formula (2) and chemical formula (3) are promising in manufacturing hole transporting type, high efficient sky-blue fluorescent devices.

The Device Efficiencies for Compounds (Chemical Formula (1), Chemical Formula (2), Chemical Formula (3) and Chemical Formula (6)) which are Used in Organic Electroluminescent Devices

Herein, the substrate was an indium tin oxide (ITO) coated glass. Devices were constructed with poly(3,4-ethylenedioxythiophene):poly(styrenesulfonate) (PEDOT:PSS) deposited onto the indium tin oxide (ITO) surface as the hole injection layer, 40 nm thickness of emitter as the organic luminescent layer (LEL), 40 nm of 2,2′,2″-(1,3,5-benzinetriyl)-tris(1-phenyl-1-H-benzimidazole) (TPBI) as the electron transport layer (ETL), 1 nm of LiF as the electron injection layer (EIL), and 150 nm of Al as the cathode, respectively, for ITO/emitter (40 nm)/TPBI (40 nm)/LiF (1 nm)/Al (150 nm) devices. Chemical formula (1), chemical formula (2) and chemical formula (3) were examined as the hole transporting type emitting layer by spin-coating process for the purpose of verifying their film ductility.

The Above-Mentioned Device Configuration Integrates with Chemical Formula (1), Chemical Formula (2) or Chemical Formula (3) to Form the Organic Electroluminescent Device. The Obtained Device Efficiencies of Each Organic Electroluminescent Device are Shown as in Table 3.

TABLE 3

| | | | η_{c}^{[c]} | |
|---|---|---|---|---|
| | | | (cd/A)/ | |
| | | | ηₚ^{[c]} | L₂₀^{[c]} |
| Device configuration^{[a]} | Em, λₘₐₓ^{[b]} (nm) | Vₒₙ^{[c]} (V) | (lm/W) | (cd/m²) |
| Chemical formula (1)/A | 444, 472 (78) | 3.2 (4.8) | 2.5/1.6 | 495 |
| Chemical formula (1)/B | 448, 472 (85) | 3.2 (6.2) | 3.4/0.7 | 667 |
| Chemical formula (1)/C | 444, 468 (64) | 3.3 (5.1) | 4.7/2.9 | 722 |
| Chemical formula (2)/A | 460, 484 (76) | 3.2 (5.0) | 3.3/2.1 | 646 |
| Chemical formula (2)/C | 460, 488 (76) | 3.7 (6.4) | 6.6/3.3 | 1307 |
| Chemical formula (3)/A | 506, 528 (113) | 2.9 (4.6) | 4.3/3.0 | 864 |
| Chemical formula (3)/C | 494, 520 (80) | 3.1 (5.0) | 9.4/5.9 | 412 |
| Chemical formula (6)/C | 460 (60) | 2.9 (4.3) | 5.4/4.2 | 595 |
| Compound 12 | 408, 424 (51) | 3.2 (5.5) | 0.7/0.4 | 132 |
| ter(9,9-spirobifluo- | | | | |
| rene)/A | | | | |

^{[a]}Device configuration: A: ITO/PEDOT:PSS/ chemical formula (1), chemical formula (2), chemical formula (3) or compound 12 (by spin-coating)/TPBI/LiF/Al;
B: ITO/chemical formula (1) (by thermo-evaporating)/TPBI/LiF/Al;
C: ITO/PEDOT:PSS/CBP: chemical formula (1), chemical formula (2), chemical formula (3) or chemical formula (6) (15 wt %)/TPBI/LiF/Al.
^{[b]}The data in parentheses correspond to full width at half-maximum.
^{[c]}η_{c}, ηₚ, and the data in parentheses for Vₒₙ and L₂₀ were measured at 20 mA/cm².

Regarding to the device configuration A, in which ter(9,9-spirobifluo-rene) acted as the emitter, it exhibited deep blue fluorescence. Further, when chemical formula (1) or chemical formula (2) acted as the emitter, it exhibited sky-blue fluorescence. And, when chemical formula (3) acted as the emitter, it exhibited bluish green fluorescence. The device configuration A with chemical formula (1), chemical formula (2) or chemical formula (3) exhibited with luminescence (L₂₀) of 495-864 cd/m² at 20 mA/cm². Their current/power efficiencies (η_{c}/ηₚ) were 2.5/1.6, 3.3/2.1, and 4.3/3.0 cdA⁻¹/lmW⁻¹ for chemical formula (1), chemical formula (2) and chemical formula (3), respectively. The η_{c} and ηₚ for chemical formula (3) are 1.3-1.7 times better than that for chemical formula (1) and chemical formula (2). Notably, chemical formula (1) and chemical formula (2) showed 3.5 to 5 times of considerably improvements in their electroluminescent efficiencies than one of the best hole transporting type blue fluorescent material (compound 12, ter(9,9-spirobifluorene)) in the same spin-coated device. It is due to the high film ductility of these oligo(cis-stilbene) trimers (chemical formula (1) and chemical formula (2)) than ter(spirobifluorene) (compound 12, ter(9,9-spirobifluorene)). Furthermore, compound 1 was fabricated by thermo-evaporation process in device configuration B and its L₂₀, η_{c}, and ηₚ were of 667 cd/m², 3.4 cd/A, and 0.7 lm/W, respectively, and are almost at the equal level in comparing with that in device configuration A. These results imply the oligo(cis-stilbene) trimers have excellent film ductility, and are capable for the device fabrication by spin-coating process. In addition, 4,4′-Bis(carbazolyl)biphenyl (CBP) was chosen as the host material, whereas chemical formula (1), chemical formula (2), chemical formula (3) and chemical formula (6) were used as guest materials (15 wt %) in the device configuration C, and they exhibited L₂₀ of 722, 1307, 412 and 595 cd/m² with η_{c}/ηₚ of 4.7/2.9, 6.6/3.3, 9.4/5.9 and 5.4/4.2 cdA⁻¹/lmW⁻¹, for chemical formula (1), chemical formula (2), chemical formula (3) and chemical formula (6), respectively. Comparing with the devices fabricated by spin-coating or by thermo-evaporation, the device with CBP doped with chemical formula (1), chemical formula (2) or chemical formula (3) can obviously improve the current efficiencies and power efficiencies. That is because CBP can transfer its electron and hole carriers efficiently to chemical formula (1), chemical formula (2), and chemical formula (3).

The spirally configured cis-stilbene/fluorene hybrid materials according to the present embodiment are a series of organic fluorescent materials, and by linking the both para-positions of stilbene moiety, dimeric or oligomeric (or quasi-oligomeric) system is formed. In the present embodiment, comparing with the devices fabricated by thermo-evaporation, the devices fabricated by spin-coating have the excellent electroluminescent efficiencies.

In summary, in the spirally configured cis-stilbene/fluorene hybrid material and the organic electroluminescent devices by using the same according to the present invention, it utilizes the spirally configured cis-stilbene/fluorene hybrid or its oligomer, such as dimer or trimer, as the core template. The material according to the present invention has excellent electroluminescent efficiency and high fluorescent quantum yield. Further, such material can be formed as a film by spin-coating, which has a lower cost than that formed by the conventional vacuum evaporation does. Moreover, the material is easily purified so as to prevent the decay of device efficiency and lifespan. In addition, the excellent film ductility of the material takes advantage in flexible device fabrication for organic electroluminescent display and light source.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A spirally configured cis-stilbene/fluorene hybrid material, comprising a structure of the following General Formula (1), 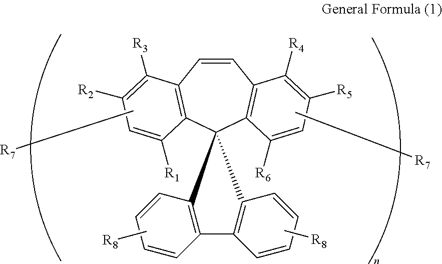
wherein, n is 1, 2 or 3, R₁ to R₆ are each independently selected from the group consisting of a hydrogen atom, halogen atom, cyano group, alkyl group, cycloalkyl group, alkoxy group, amino group, haloalkyl group, thioalkyl group, silyl group, alkenyl group and aromatic ring group, R₈ is a hydrogen atom, tert-butyl group or aromatic ring group,
when n is 1, is 9,9′-spirobifluorene group, diarylamino group or 2-diarylamino-spirobifluorene group,
when n is 2 or 3, R₇ is a hydrogen atom, diarylamino group or 2-diarylamino-spirobifluorene group.

2. The spirally configured cis-stilbene/fluorene hybrid material of claim 1, wherein the alkyl group is selected from the group consisting of a substituted or unsubstituted straight-chain alkyl group with the carbon number of 1 to 6, and a substituted or unsubstituted branched-chain alkyl group with the carbon number of 3 to 6, the cycloalkyl group is a substituted or unsubstituted cycloalkyl group with the carbon number of 3 to 6, the alkoxy group is selected from the group consisting of a substituted or unsubstituted straight-chain alkoxy group with the carbon number of 1 to 6, and a substituted or unsubstituted branched-chain alkoxy group with the carbon number of 3 to 6, the amino group is selected from the group consisting of secondary amino group and tertiary amino group, the secondary amino group is an amino group having one aromatic ring substituent or having one C1-C6 straight-chain alkyl, branch-chain alkyl, or non-aromatic ring, the tertiary amino group is an amino group having two independent aromatic ring substituents or having two independent C1-C6 straight-chain alkyl, branch-chain alkyl, or non-aromatic ring, the haloalkyl group is selected from the group consisting of a substituted or unsubstituted straight-chain haloalkyl group with the carbon number of 1 to 6, and a substituted or unsubstituted branched-chain haloalkyl group with the carbon number of 3 to 6, the thioalkyl group is selected from the group consisting of a substituted or unsubstituted straight-chain thioalkyl group with the carbon number of 1 to 6, and a substituted or unsubstituted branched-chain thioalkyl group with the carbon number of 3 to 6, the silyl group is selected from the group consisting of a substituted or unsubstituted straight-chain silyl group with the carbon number of 1 to 6, and a substituted or unsubstituted branched-chain silyl group with the carbon number of 3 to 6, the alkenyl group is selected from the group consisting of a substituted or unsubstituted straight-chain alkenyl group with the carbon number of 2 to 6, and a substituted or unsubstituted branched-chain alkenyl group with the carbon number of 3 to 6.

3. The spirally configured cis-stilbene/fluorene hybrid material of claim 1, being represented by following chemical formula (1), chemical formula (2), chemical formula (3), chemical formula (4), chemical formula (5) or chemical formula (6).

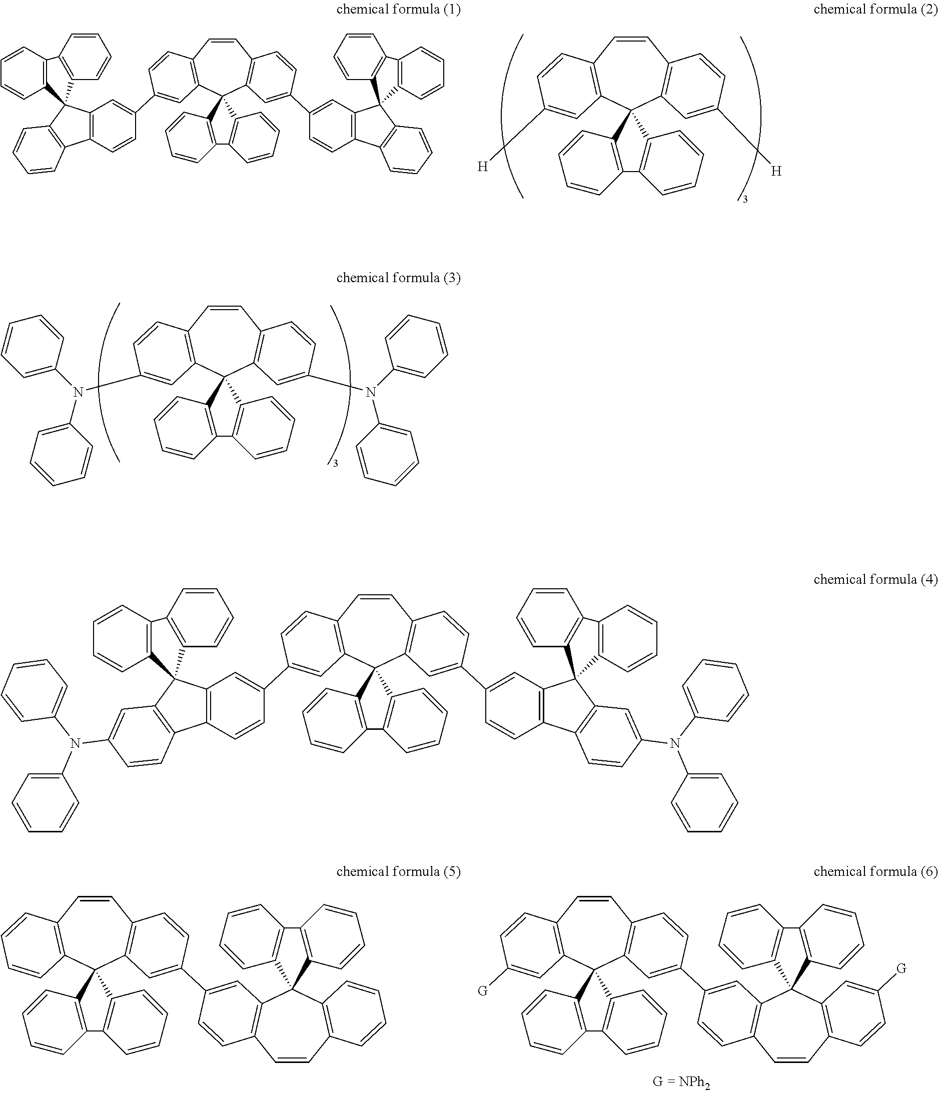

4. The spirally configured cis-stilbene/fluorene hybrid material of claim 1, having glass transition temperatures ranged from 221° C. to 231° C.

5. The spirally configured cis-stilbene/fluorene hybrid material of claim 1, having decomposition temperatures ranged from 491° C. to 535° C.

6. The spirally configured cis-stilbene/fluorene hybrid material of claim 1, having oxidation potentials ranged from +0.42V to +0.86V and reduction potentials ranged from −2.44V to −2.64V.

7. An organic electroluminescent device, comprising:
a first electrode layer;
a second electrode layer; and
an organic luminescent unit, deposited between the first electrode layer and the second electrode layer, wherein the organic luminescent unit has at least a spirally configured cis-stilbene/fluorene hybrid material as shown in General Formula (1), 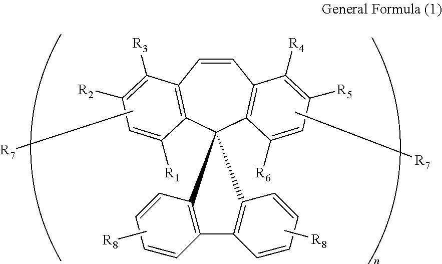
wherein, n is 1, 2 or 3, R₁ to R₆ are each independently selected from the group consisting of a hydrogen atom, halogen atom, cyano group, alkyl group, cycloalkyl group, alkoxy group, amino group, haloalkyl group, thioalkyl group, silyl group, alkenyl group and aromatic ring group, R₈ is a hydrogen atom, tert-butyl group or aromatic ring group,
when n is 1, is 9,9′-spirobifluorene group, diarylamino group or 2-diarylamino-spirobifluorene group,
when n is 2 or 3, R₇ is a hydrogen atom, diarylamino group or 2-diarylamino-spirobifluorene group.

8. The organic electroluminescent device of claim 7, wherein the alkyl group is selected from the group consisting of a substituted or unsubstituted straight-chain alkyl group with the carbon number of 1 to 6, and a substituted or unsubstituted branched-chain alkyl group with the carbon number of 3 to 6, the cycloalkyl group is a substituted or unsubstituted cycloalkyl group with the carbon number of 3 to 6, the alkoxy group is selected from the group consisting of a substituted or unsubstituted straight-chain alkoxy group with the carbon number of 1 to 6, and a substituted or unsubstituted branched-chain alkoxy group with the carbon number of 3 to 6, the amino group is selected from the group consisting of secondary amino group and tertiary amino group, the secondary amino group is an amino group having one aromatic ring substituent or having one C1-C6 straight-chain alkyl, branch-chain alkyl, or non-aromatic ring, the tertiary amino group is an amino group having two independent aromatic ring substituents or having two independent C1-C6 straight-chain alkyl, branch-chain alkyl, or non-aromatic ring, the haloalkyl group is selected from the group consisting of a substituted or unsubstituted straight-chain haloalkyl group with the carbon number of 1 to 6, and a substituted or unsubstituted branched-chain haloalkyl group with the carbon number of 3 to 6, the thioalkyl group is selected from the group consisting of a substituted or unsubstituted straight-chain thioalkyl group with the carbon number of 1 to 6, and a substituted or unsubstituted branched-chain thioalkyl group with the carbon number of 3 to 6, the silyl group is selected from the group consisting of a substituted or unsubstituted straight-chain silyl group with the carbon number of 1 to 6, and a substituted or unsubstituted branched-chain silyl group with the carbon number of 3 to 6, the alkenyl group is selected from the group consisting of a substituted or unsubstituted straight-chain alkenyl group with the carbon number of 2 to 6, and a substituted or unsubstituted branched-chain alkenyl group with the carbon number of 3 to 6.

9. The organic electroluminescent device of claim 7, being represented by following chemical formula (1), chemical formula (2), chemical formula (3), chemical formula (4), chemical formula (5) or chemical formula (6).

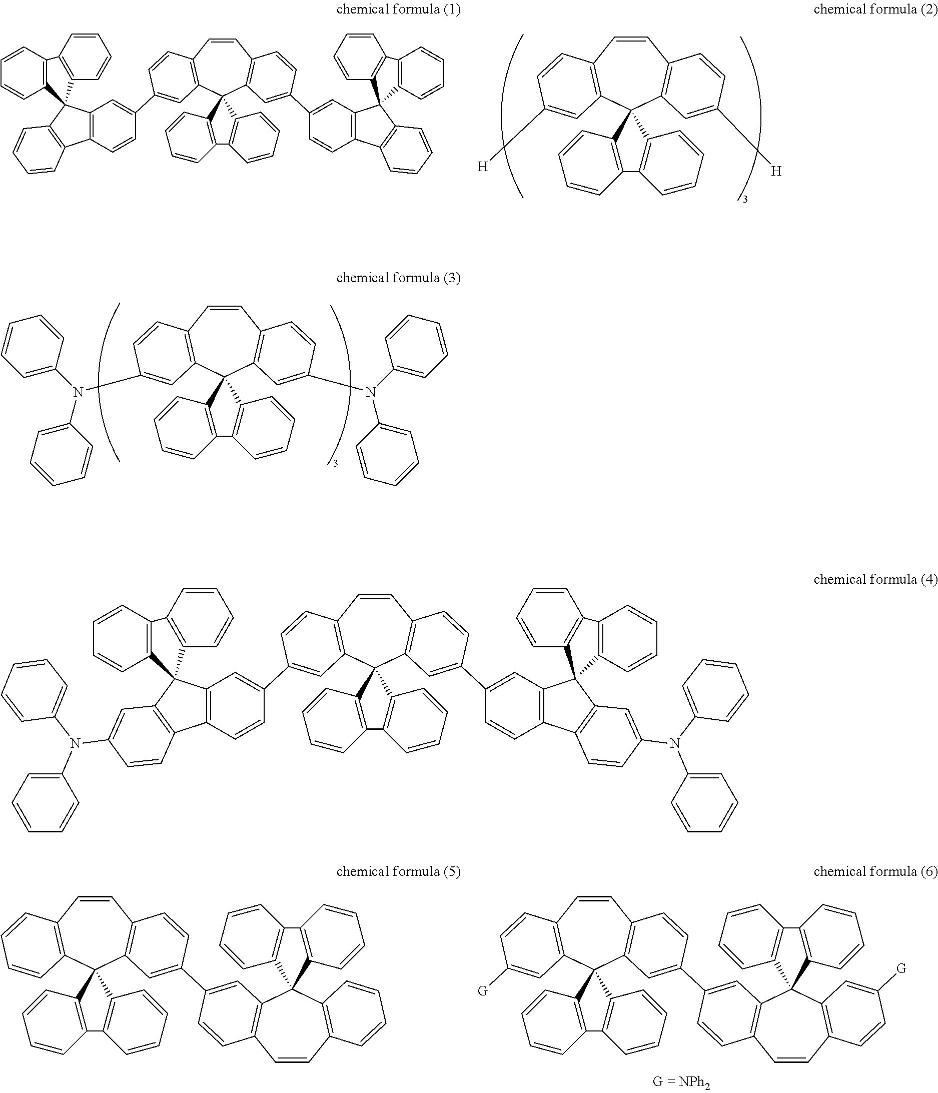

10. The organic electroluminescent device of claim 7, wherein the organic luminescent unit comprises an organic luminescent layer.

11. The organic electroluminescent device of claim 10, wherein the organic luminescent unit further comprises a hole transport layer and an electron transport layer, and the organic luminescent layer is deposited between the hole transport layer and the electron transport layer.

12. The organic electroluminescent device of claim 10, wherein the organic luminescent unit further comprises a hole injection layer, a hole transport layer, an electron transport layer and an electron injection layer, and the hole transport layer, the organic luminescent layer and the electron transport layer are sequentially deposited between the hole injection layer and the electron injection layer.

13. The organic electroluminescent device of claim 10, wherein the organic luminescent layer comprises the spirally configured cis-stilbene/fluorene hybrid material.

14. The organic electroluminescent device of claim 10, wherein the organic luminescent layer comprises a host material and a guest material, and the host material or the guest material comprises the spirally configured cis-stilbene/fluorene hybrid material.

15. The organic electroluminescent device of claim 14, wherein the content of the guest material in the organic luminescent layer is between 3 wt % to 15 wt %.

16. The organic electroluminescent device of claim 15, wherein the content of the guest material in the organic luminescent layer is 15 wt %.
